(19) United States
(12) Patent Application Publication
(10) Pub. No.: US 2019/0192299 A1
(43) Pub. Date: Jun. 27, 2019

(54) DEVICE AND METHOD FOR MANUFACTURING ARTIFICIAL SOLID BONE

(71) Applicant: KOLN 3D TECHNOLOGY (MEDICAL) LIMITED, Hong Kong (CN)

(72) Inventor: Wing Fung Edmond YAU, Hong Kong (CN)

(21) Appl. No.: 16/331,922

(22) PCT Filed: Sep. 7, 2017
(86) PCT No.: PCT/IB2017/055386

§ 371 Date: Mar. 8, 2019

(30) Foreign Application Priority Data
Sep. 8, 2016 (HK) 16110702.0

(51) Int. Cl. A61F 2/30; A61F 2/28; B33Y 10/00; B33Y 70/00; B33Y 80/00; B22F 3/105; B22F 3/24

(52) U.S. Cl. CPC A61F 2/30; B22F 2301/15; A61F 2/30771; B33Y 10/00; B33Y 70/00; B33Y 80/00; B22F 3/1055; B22F 3/24; A61F 2002/3097; A61F 2002/30985; A61F 2310/00029; A61F 2310/00059; A61F 2310/00071; A61F 2310/00101; A61F 2310/00137; A61F 2002/30006; A61F 2002/30968; B22F 2003/247; A61F 2/28

(57) ABSTRACT

A device and method for manufacturing artificial solid bone by metal 3D printing technology comprises: a metal 3D printing technology is used and preferably with Co—Cr alloy and laser sintering to form a solid bone with a specific change in shape and density; a synchronous cutting operation performed on approximately 80% of the preferred surface of the solid bone while the metal 3D printer unit is printing the solid bone, to make the solid bone have the following surface roughness: Ry<1˜2 μm or less; and a synchronous polishing operation performed on at least one joint surface of the solid bone, to make the joint surface have the following surface roughness: Class A4=Ra0.063 μm or less.

### TECHNICAL FIELD

The present disclosure relates to an artificial solid bone, and more particularly relates to a device and method for manufacturing an artificial solid bone by a metal 3D printing technology.

### BACKGROUND

In the treatment with artificial solid bone (such as ankle), artificial solid bone made of plastic is used to replace solid bone of the human body. Since solid bone bears greater weight and force, a plastic artificial bone is usually used for 8-12 months, and then needs surgery to be replaced.

In addition, joint surfaces of solid bone should be smooth enough because they have to bear greater gravity and often friction with other bones, in which a surface roughness (Ry) needs to reach 1˜2 μm or less. Therefore, the artificial solid bone manufactured with the existing metal 3D printing method is not applicable.

The existing medical method using artificial bones is not an ideal solution because it poses risks, pain, and additional costs to patients.

### SUMMARY

A method for manufacturing artificial solid bone is provided, which comprises:

A metal 3D printing technology is used and preferably with Co—Cr alloy and direct metal laser sintering to form a solid bone with a specific shape and density;

A synchronous cutting operation performed on approximately 80% of the preferred surface of the solid bone after the metal 3D printer unit prints and forms the solid bone, to make the solid bone have the following surface roughness: Ry<1˜2 μm or less;

A synchronous polishing operation performed on at least one contact surface of the solid bone, to make the joint surface have the following surface roughness: Class A4=Ra0.063 μm or less.

In some embodiments, the solid bone printed and formed includes an extension part formed at the top and/or bottom of the solid bone, which facilitates a subsequent processing operation of the solid bone, the extension part is preferably a cylinder part with a diameter of 8 mm and a length of 8˜10 mm, and an axis of the cylinder part is parallel and/or coincident with a center axis of the solid bone; and/or the cylinder part is configured to be coupled with a processing machine for subsequent processing operations for required processing operations.

In some other embodiments, the solid bone comprises a first density part and a second density part whose density is lower than the first density part, wherein, the first density part may preferably achieve a density higher than the second density part by performing additional sintering operations; and/or the second density part is preferably configured to have a grid structure to achieve a density lower than the first density part; and/or the first density part is preferably located peripherally to the second density part; the first density part preferably has a relative density of 99.5% or more and the second density part preferably has a relative density of 90% or more.

In some embodiments, the solid bone is a foot-ankle bone; and/or the Co—Cr alloy includes Co—Cr—Mo and/or Co—Cr—W—Ni.

A device for manufacturing artificial solid bone is also provided in the present disclosure, comprising:

A metal 3D printer unit that is preferably configured to be with Co—Cr alloy and direct metal laser sintering to form a solid bone with a specific shape and density;

A cutting unit operationally connected to the metal 3D printer unit performs a synchronous cutting operation on approximately 80% of the surface of the solid bone while the metal 3D printer unit is printing the solid bone, to make the solid bone have the following surface roughness: Ry<1˜2 μm or less;

A polishing unit operationally connected to the cutting unit performs a synchronous polishing operation on at least one contact surface of the solid bone, to make the joint surface have the following surface roughness: Class A4=Ra0.063 μm or less.

In some embodiments, the solid bone printed and formed by the metal 3D printer unit includes an extension part formed at the top and/or bottom of the solid bone, which facilitates subsequent processing operations of the solid bone, the extension part is preferably a cylinder part, comprising a cylinder part with a diameter of 8 mm and a length of 8˜10 mm. An axis of the cylinder part is parallel and/or coincident with a center axis of the solid bone; and/or the cylinder part is configured to couple with the cutting unit and/or the polishing unit for processing operations.

In some embodiments, the solid bone formed by the metal 3D printer unit comprises a first density part and a second density part whose density is lower than the first density part, wherein the first density part may preferably achieve a density higher than the second density part by performing additional sintering operations; and/or the second density part is preferably configured to have a grid structure to achieve a density lower than the first density part; and/or the first density part is preferably located peripherally to the second density part; the first density part preferably has a relative density of 99.5% or more and the second density part preferably has a relative density of 90% or more.

### BRIEF DESCRIPTION OF THE DRAWINGS

The present disclosure is described by examples with reference to the attached drawings:

FIG. 1a is a schematic diagram of one example of an artificial solid bone in which the disclosed embodiments may be implemented;

FIG. 1b is a schematic diagram of another example of an artificial solid bone in which the disclosed embodiments may be implemented;

FIG. 2a is a schematic diagram of another example of an artificial solid bone in which the disclosed embodiments may be implemented;

FIG. 2b is a schematic part diagram obtained along an A-A line of FIG. 2a; and

FIG. 3 is a schematic diagram of another example of an artificial solid bone with a grid structure in which the disclosed embodiments may be implemented.

### DETAILED DESCRIPTION

Some preferred embodiments of the present disclosure are described below in conjunction with the attached drawings in order to illustrate the technical solutions in detail.

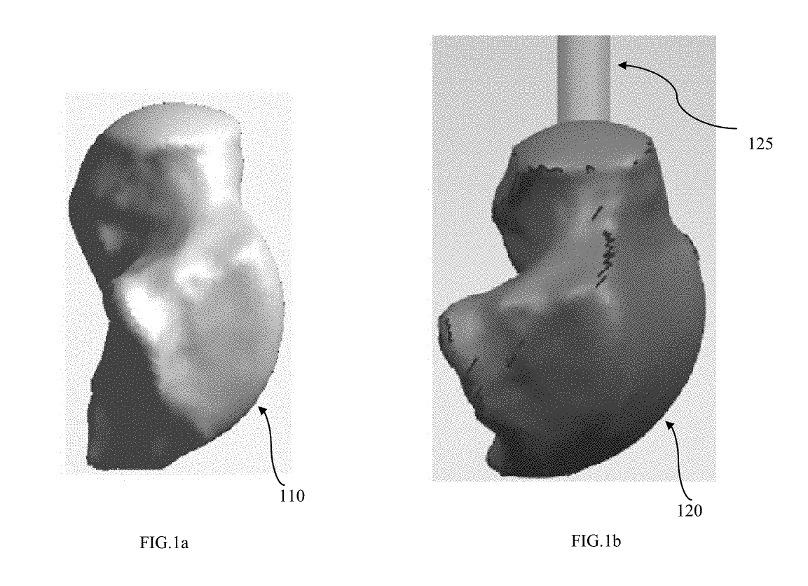

In the exemplary embodiment illustrated in FIG. 1a, which is a schematic diagram of one example of an artificial solid bone 110, the solid bone is preferably a foot-ankle bone. A method for manufacturing the artificial solid bone comprises: a metal 3D printer unit that is preferably configured to be with Co—Cr alloy and laser sintering to form a solid bone with a specific shape and density; a synchronous cutting operation performed on approximately 80% of the surface of the solid bone while the metal 3D printer unit is printing the solid bone, to make the solid bone have the following surface roughness: Ry<1˜2 μm or less; and a synchronous polishing operation performed on at least one joint surface of the solid bone, to make the contact surface have the following surface roughness: Class A4=Ra0.063 μm or less.

In some embodiments, the Co—Cr alloy includes Co—Cr—Mo and/or Co—Cr—W—Ni.

In the exemplary embodiment illustrated in FIG. 1b, which is a schematic diagram of one example of an artificial solid bone 120, the solid bone printed and formed comprises an extension part 125 formed at the top and/or bottom of the solid bone, which facilitates a subsequent processing operation of the solid bone, the extension part is preferably a main body part, comprising a cylinder part with a diameter of 8 mm and a length of 8˜10 mm, also a triangular column part, a square column part, a multilateral column part, and/or a composite column part or a special-shaped column part, wherein a central axis of the column part is parallel and/or coincident with a central axis of the solid bone; and/or the cylinder part is configured to be coupled to the processing machine for processing operations.

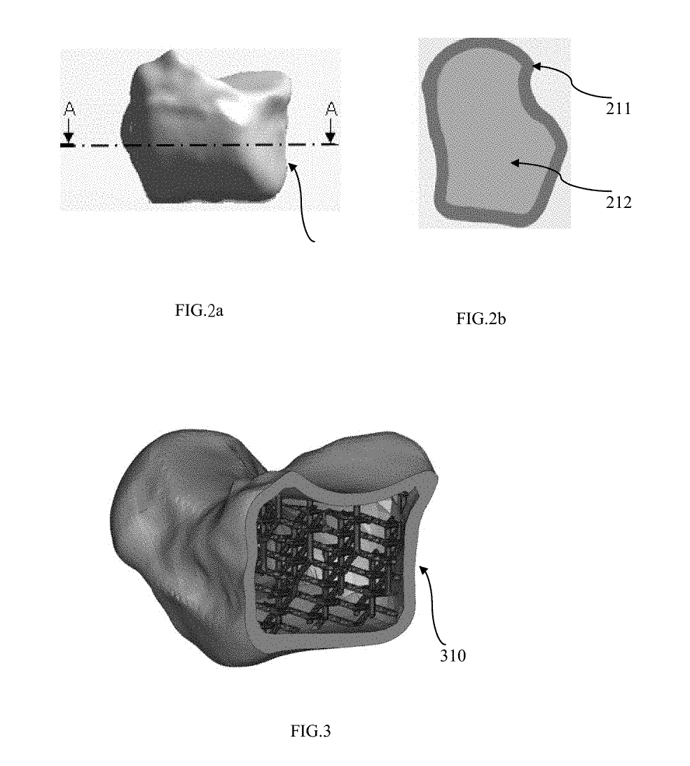

In the exemplary embodiment illustrated in FIGS. 2a-2b, of which FIG. 2a is a schematic diagram of another example of an artificial solid bone 210 and FIG. 2b is a schematic part diagram obtained along an A-A line of FIG. 2a, the solid bone comprises a first density part 211 and a second density part 212 whose density is lower than the first density part 211, wherein the first density part 211 may preferably achieve a density higher than the second density part 212 by performing additional sintering operations; and/or the first density part 211 is preferably located peripherally to the second density part 212; the first density part 211 preferably has a relative density of 99.5% or more and the second density part 212 preferably has a relative density of 90% or more.

In the exemplary embodiment illustrated in FIG. 3, which is a schematic diagram of another example of an artificial solid bone 310 with a grid structure, the solid bone also includes a first density part and a second density part whose density is lower than the first density part, wherein the first density part may preferably achieve a density higher than the second density part by performing additional sintering operations; alternatively, the second density part may be preferably configured to have a grid structure to achieve a density lower than the first density part; the first density part is preferably located peripherally to the second density part; the first density part preferably has a relative density of 99.5% or more and the second density part preferably has a relative density of 90% or more.

A device for manufacturing artificial solid bone is also provided in the present disclosure, comprising:

A metal 3D printer unit that is preferably configured to be with Co—Cr alloy and laser sintering to form a solid bone with a specific shape and density;

A cutting unit operationally connected to the metal 3D printer unit performs a synchronous cutting operation on approximately 80% of the surface of the solid bone while the metal 3D printer unit is printing the solid bone, to make the solid bone have the following surface roughness: Ry<1˜2 μm or less;

A polishing unit operationally connected to the cutting unit performs a synchronous polishing operation performed on at least one contact surface of the solid bone, to make the joint surface have the following surface roughness: Class A4=Ra0.063 μm or less.

In some embodiments, the solid bone printed and formed by the metal 3D printer unit includes an extension part formed at the top and/or bottom of the solid bone, which facilitates subsequent processing operations of the solid bone, the extension part is preferably a cylinder part with a diameter of 8 mm and a length of 8˜10 mm. An axis of the cylinder part is parallel and/or coincident with a center axis of the solid bone; and/or the cylinder part is configured to couple with the cutting unit and/or the polishing unit to perform the processing operations required for the main body part or particular position of the solid bone. In some embodiments, the extension part may preferably be positioned relative to a particular part or position to be processed to facilitate the cutting/polishing operation of the cutting and/or polishing units.

In some embodiments, the solid bone formed by the metal 3D printer unit comprises a first density part and a second density part whose density is lower than the first density part, wherein the first density part may preferably achieve a density higher than the second density part by performing additional sintering operations; and/or the second density part is preferably configured to have a grid structure to achieve a density lower than the first density part, the grid structure is uniformly distributed or concentrated in a particular region of the second density part, such that the second density part has a predetermined density or weight; and/or the first density part is preferably located peripherally to the second density part; the first density part preferably has a relative density of 99.5% or more and the second density part preferably has a relative density of 90% or more.

The metal 3D patented printing technology used in the present disclosure adopts Co—Cr alloy to replace the damaged/lost bone, because the Co—Cr alloy has the following main characteristics:

Its main chemical components include Co—Cr—Mo, Co—Cr—W—Ni and the like, leading its corrosion resistance dozens of times higher than that of stainless steel, and generally without obvious tissue reaction.

Used in artificial hip joint, the Co—Cr alloy has excellent friction resistance and strong bearing capacity, which is suitable to be used as material of implant and meets anti-wear function required by the solid bone.

According to the method for manufacturing artificial solid bone described in the present disclosure, after several steps of laser sintering, a built-in processing unit immediately performs processing operation. The two processes iterate to ensure that most positions of the solid bone can reach surface roughness Ry<1˜2 μm or less, which is in line with the demands of the solid bone.

A metal 3D printer unit described in the present disclosure adopts metal powder laser shaping technology, wherein its laser melting power is about 400 W, a cutting unit operably connected with the metal 3D printer unit is preferably a high speed milling unit with a speed of its milling cutter about 45,000/min.

In some embodiments, a metal 3D printing technology in accordance with the present disclosure adopts a technical proposal composed of Co—Cr (Cobalt-Chrome) alloy and metal powder direct laser sintering/direct metal laser sintering (DMLS) technology and synchronous metal cutting function/parts to reduce post-processing time. A combination of laser sintering and synchronous cutting enables about 80% or more of surface of a product or solid bone to be cut simultaneously, and obtains a surface roughness Ry<1˜2 μm or less, wherein a further polishing on surface or position of the Co—Cr solid bone (such as foot-ankle bone) that may contact with other bone 3 can make the related surface or position achieve a surface roughness about Class A4=Ra0.063 μm or less to serve as a joint.

The technical proposal for manufacturing artificial solid bone in accordance with the present disclosure can make lifespan of metal ankle bone increased to up to 8-10 years, thus reducing surgical risk and lessen surgical procedures to existing patients who need to replace plastic ankle bone each year. Surgeries of replacing plastic bone every year require a lot of resources; for example, time of doctors and medical personnel and surgical rooms, etc., therefore, the current technology causes high cost and inconvenience to users. The above problems can be solved by the technical proposal of the present disclosure, so that more patients can benefit from the 3D printing technology.

Although different examples or embodiments have been described in the present disclosure, it should be understood that they are used to illustrate rather than limit the scope of protection. It should be understood that parts or components of different exemplary embodiments may, where appropriate, be combined and/or mixed together to form other variants without losing generality.

1. The present disclosure relates to a method for manufacturing artificial solid bone, which comprises:
a metal 3D printing technology is used and preferably with Co—Cr alloy and direct metal laser sintering to form a solid bone with a specific shape and density;
a synchronous cutting operation performed on approximately 80% of the surface of the solid bone while the metal 3D printer unit is printing the solid bone, to make the solid bone have the following surface roughness: Ry<1˜2 μm; and
a synchronous polishing operation performed on at least one contact surface of the solid bone, to make the joint surface have the following surface roughness: Class A4=Ra0.063 μm or less.

2. The method for manufacturing artificial solid bone according to claim 1, wherein:
the solid bone printed and formed by the metal 3D printer unit includes an extension part formed at the top and/or bottom of the solid bone, which facilitates subsequent processing operations of the solid bone, the extension part is preferably a cylinder part, comprising a cylinder part with a diameter of 8 mm and a length of 8˜10 mm. An axis of the cylinder part is parallel and/or coincident with a center axis of the solid bone; and/or the cylinder part is configured to couple with the processing machine for subsequent processing operations to carry out the required processing operations.

3. The method for manufacturing artificial solid bone according to claim 1, wherein:
the solid bone comprises a first density part and a second density part whose density is lower than the first density part, wherein the first density part may preferably achieve a density higher than the second density part by performing additional sintering operations; and/or the second density part is preferably configured to have a grid structure to achieve a density lower than the first density part; and/or the first density part is preferably located peripherally to the second density part; the first density part preferably has a relative density of 99.5% or more and the second density part preferably has a relative density of 90% or more.

4. The method for manufacturing artificial solid bone according to claim 1, wherein:
the solid bone is a foot-ankle bone; and/or the Co—Cr alloy includes Co—Cr—Mo and/or Co—Cr—W—Ni.

5. The method for manufacturing artificial solid bone according to claim 1, wherein: the solid bone is manufactured by a manufacturing device, and the manufacturing device comprises:
a metal 3D printer unit that is preferably configured to sinter with a Co—Cr alloy and a direct metal laser to form a solid bone with a specific shape and density;
a cutting unit operationally connected to the metal 3D printer unit performs a synchronous cutting operation on approximately 80% of the preferred surface of the solid bone while the metal 3D printer unit is printing the solid bone, to make the solid bone have the following surface roughness: Ry<1˜2 μm;
a polishing unit operationally connected to the cutting unit performs a synchronous polishing operation on at least one joint surface of the solid bone, to make the joint surface have the following surface roughness: Class A4=Ra0.063 μm or less.

6. The method for manufacturing artificial solid bone according to claim 5, wherein:
the solid bone printed and formed by the metal 3D printer unit includes an extension part formed at the top and/or bottom of the solid bone, which facilitates subsequent processing operations of the solid bone, the extension part is preferably a cylinder part, comprising a cylinder part with a diameter of 8 mm and a length of 8˜10 mm. An axis of the cylinder part is parallel and/or coincident with a center axis of the solid bone; and/or the cylinder part is configured to couple with the cutting unit and/or the polishing unit for processing operations.

7. The method according to claim 5, wherein:
the solid bone comprises a first density part and a second density part whose density is lower than the first density part, wherein the first density part may preferably achieve a density higher than the second density part by performing additional sintering operations; and/or the second density part is preferably configured to have a grid structure to achieve a density lower than the first density part; and/or the first density part is preferably located peripherally to the second density part; the first density part preferably has a relative density of 99.5% or more and the second density part preferably has a relative density of 90% or more.

8. The method for manufacturing artificial solid bone according to claim 5, wherein:
the solid bone formed by the metal 3D printer unit is a foot-ankle bone; and/or the Co—Cr alloy includes Co—Cr—Mo and/or Co—Cr—W—Ni.
